(19) United States
(12) Patent Application Publication
(10) Pub. No.: US 2025/0213804 A1
(43) Pub. Date: Jul. 3, 2025

(54) AUTOMATIC DRUG DELIVERY DEVICE FOR INTELLIGENT INHALATION MEDICATION

(71) Applicant: XEEK (Xiamen) Medical Equipment Co,. Ltd., Xiamen (CN)

(72) Inventors: Jingping ZHENG, Xiamen (CN); Mingjun He, Xiamen (CN); Yougan Xie, Xiamen (CN); Yicong Huang, Xiamen (CN); Wenxin Zheng, Xiamen (CN); Yi He, Xiamen (CN)

(21) Appl. No.: 18/660,622

(22) Filed: May 10, 2024

(30) Foreign Application Priority Data
Dec. 29, 2023 (CN) 202311845790.9

(51) Int. Cl. A61M 15/00; A61M 11/00

(52) U.S. Cl. CPC A61M 15/0093; A61M 11/006; A61M 15/009; A61M 2205/103; A61M 2205/3334; A61M 2205/50

(57) ABSTRACT

The disclosure discloses an automatic drug delivery device for intelligent inhalation medication, including a drug inhaler, a drug inhalation action detection unit, a main control unit and an automatic pressing unit. The drug inhaler is a drug inhaler capable of drug delivery by pressing. The drug inhalation action detection unit is configured to detect whether the drug inhaler is subjected to a drug inhalation action and transmit a drug inhalation action signal to the main control unit. The main control unit is configured to correspondingly drive the automatic pressing unit to press a canister of the drug inhaler for drug delivery according to the received drug inhalation action signal.

### CROSS-REFERENCE TO RELATED APPLICATIONS

This application claims priority to Chinese Patent Application 202311845790.9, filed on Dec. 29, 2023, which is incorporated herein by reference.

### TECHNICAL FIELD

The disclosure belongs to the field of medication management, and particularly relates to an automatic drug delivery device for intelligent inhalation medication.

### BACKGROUND

Many respiratory diseases, such as asthma, COPD, cystic fibrosis and bronchiectasis, require patients to rely on long-term medication. Drug inhalers are often used to achieve medication during drug therapy. The commonly used drug inhalers are pressurized metered-dose inhalers (pMDIs) and dry powder inhalers (DPIs). The pMDI does not rely on the patient's active inhalation to generate energy to depolymerize the drug powder to output drug particles, and therefore requires a low inspiratory flow rate during use. Moreover, the internal resistance of the pMDI is very low, which can hardly affect the patient's inspiratory flow rate, so the patient can achieve the required flow rate by gently inhaling. An excessive inspiratory flow rate will increase the drug deposition in the oropharynx. Therefore, when using a pMDI, the patient needs to inhale slowly, steadily and deeply. The inhalation time is 2 s to 3 s for children and 4 s to 5 s for adults. Such an inhalation process can control the inspiratory flow rate within the optimal range of using the pMDI, i.e., 30 L/min. It is necessary to press the canister of the pMDI to output the drug particles at the start of inhalation, so it is required to coordinate the pressing action with the inhalation action, i.e., good coordination between the hand and the mouth is required.

The pMDI does not need a high inspiratory pressure due to its small internal resistance, and is convenient to carry, capable of multiple dose administration and low in price, so it is widely used for patients with asthma and COPD. The investigation of Guangzhou Institute of Respiratory Diseases shows that of the 309 outpatients who used aerosol for more than half a year, only 38% could inhale correctly, and those incapable of correct inhalation were mainly due to the incoordination between the hand and the mouth. In order to increase the lung deposition percentage of the drug, it is required to press the canister while inhaling the drug. Generally, it is required to press the canister for 0 s to 0.5 s. This requires good coordination between the hand and the mouth. When the canister is pressed manually, it is very likely to press the canister too early or too late, which reduces the lung deposition percentage of the drug and affects the effect of drug therapy.

In addition, upon testing, a force of up to 2 kg to 4 kg (20 N to 40 N) is required to press the canister, and the user generally presses the canister with a finger. This is very unfriendly to the elderly and children, and it is difficult to achieve coordination between the hand and the mouth.

### SUMMARY

An objective of the disclosure is to provide an automatic drug delivery device for intelligent inhalation medication so as to solve the above technical problems.

In order to achieve the above objective, the disclosure adopts the following technical solution: an automatic drug delivery device for intelligent inhalation medication includes a drug inhaler, a drug inhalation action detection unit, a main control unit and an automatic pressing unit. The drug inhaler is a drug inhaler capable of drug delivery by pressing. The drug inhalation action detection unit is configured to detect whether the drug inhaler is subjected to a drug inhalation action and transmit a drug inhalation action signal to the main control unit. The main control unit is configured to correspondingly drive the automatic pressing unit to press a canister of the drug inhaler for drug delivery according to the received drug inhalation action signal.

Further, the drug inhalation action detection unit is realized by a flow rate detection unit. The flow rate detection unit is configured to detect an inspiratory flow rate of the drug inhaler and transmit an inspiratory flow rate signal to the main control unit, and the main control unit is configured to correspondingly drive the automatic pressing unit to press the canister of the drug inhaler for drug delivery according to the received inspiratory flow rate signal.

Furthermore, the drug inhaler includes an actuator and the canister. The actuator is provided with a chamber having a first end opening and a second end opening. The canister is mounted in the chamber from the first end opening, and the second end opening is a drug inhalation opening.

Furthermore, the flow rate detection unit is realized by a pressure sensor, and the flow rate detection unit and the main control unit are arranged on the actuator; or the flow rate detection unit, the main control unit and the automatic pressing unit are provided as a one-piece structure.

Further, the automatic pressing unit is detachably fixed to the actuator; or the automatic pressing unit and the actuator are of a one-piece structure.

Furthermore, the automatic pressing unit and the actuator are detachably connected by means of a rotary snap-fit, a threaded connection or a fastening snap-fit.

Further, the automatic pressing unit is provided with first connecting contacts, and the actuator is provided with second connecting contacts. The automatic pressing unit is electrically connected to the main control unit by means of contacting and electrical connection between the first connecting contacts and the second connecting contacts.

Further, when the flow rate detection unit and the main control unit are arranged on the actuator, the actuator is provided with a mounting cavity, the flow rate detection unit and the main control unit are mounted in the mounting cavity, the actuator is provided with a flow rate detection channel, the flow rate detection channel communicates with the mounting cavity and the chamber at the first end opening, and the flow rate detection unit is configured to detect a flow rate of the flow rate detection channel.

Further, the automatic pressing unit is realized by a motor.

Furthermore, the automatic pressing unit includes the motor, a reduction box and a screw rod. An output shaft of the motor is connected to the screw rod by means of the reduction box, and the screw rod is configured to push and press the canister of the drug inhaler for drug delivery.

Further, the main control unit controls a load current ITrip outputted by a driving circuit of the motor to be not greater than ITripMAX, and a magnitude of ITripMAX determines a maximum output torque of the motor. An ITripMAX value of the maximum output torque is obtained by means of a laboratory test, and a magnitude of an output voltage of a control lead of the main control unit is calculated based on this value.

The disclosure has the following beneficial technical effects:

The disclosure can realize automatic pressing of the canister for drug delivery, and can effectively solve the problem that the children and the elderly do not have sufficient pressing force when using a pressurized drug inhaler and the problem that it is likely to press the canister too early or too late during drug inhalation, thereby effectively enhancing the effect of drug therapy and the medication adherence and improving the quality of life.

### BRIEF DESCRIPTION OF DRAWINGS

To describe the technical solutions in the examples of the disclosure more clearly, the following briefly describes the accompanying drawings required for describing the examples. Apparently, the accompanying drawings in the following description show merely some examples of the disclosure, and a person of ordinary skill in the art may still derive other drawings from these accompanying drawings without creative efforts.

FIG. 1 is a structural view according to a specific example of the disclosure;

FIG. 2 is a structural view from another viewing angle according a specific example of the disclosure;

FIG. 3 is a partial exploded view according a specific example of the disclosure;

FIG. 4 is a partial exploded view from another viewing angle according a specific example of the disclosure;

FIG. 5 is a cutaway view according a specific example of the disclosure;

FIG. 6 is another cutaway view according a specific example of the disclosure;

FIG. 7 is a structural view of FIG. 6 from another viewing angle;

FIG. 8 is a structural view of an actuator according a specific example of the disclosure; and

FIG. 9 is a workflow block diagram according a specific example of the disclosure.

### DESCRIPTION OF EMBODIMENTS

To further illustrate the examples, the accompanying drawings are provided in the disclosure. These accompanying drawings are a part of the contents disclosed in the disclosure that are mainly used to illustrate the exampled, and can be used in conjunction with the related descriptions in the specification to explain the operation principle of the examples. With reference to these contents, those of ordinary skills in the art should be able to understand other possible implementations and advantages of the disclosure. Components in the drawings are not drawn to scale, and like component symbols are usually used to represent like components.

The disclosure will be further described in conjunction with the accompanying drawings and specific implementations.

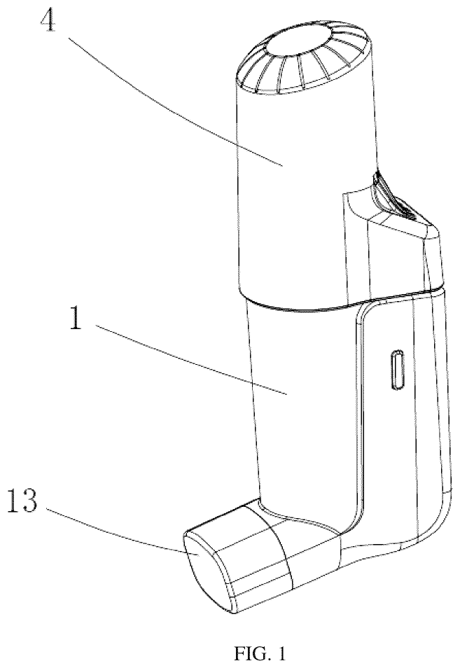

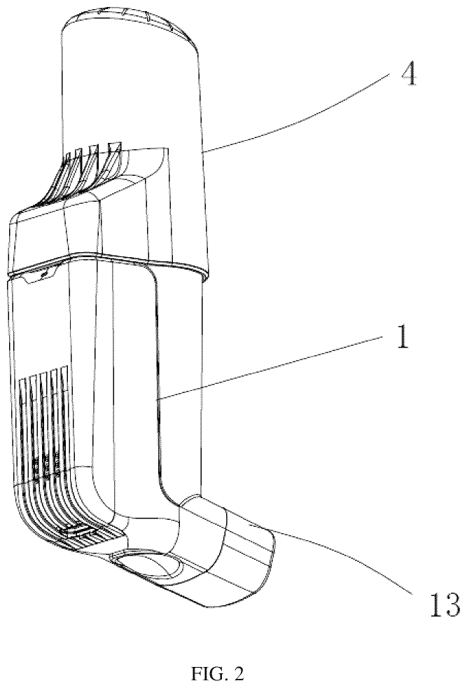

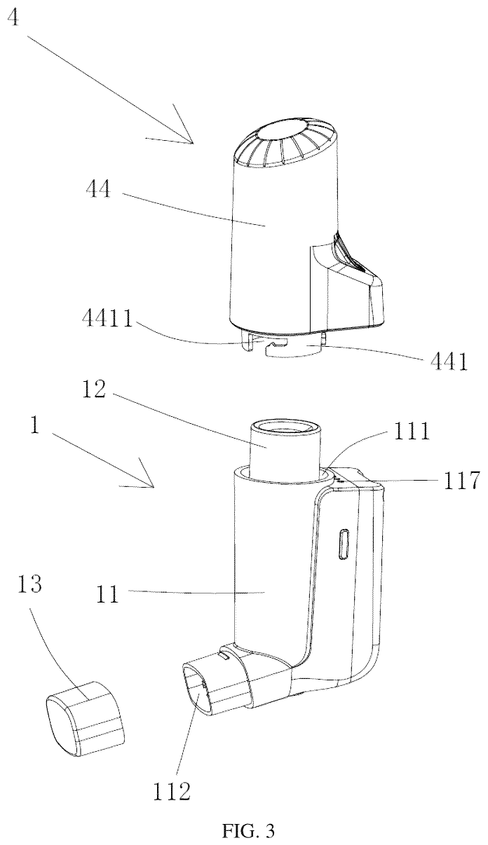

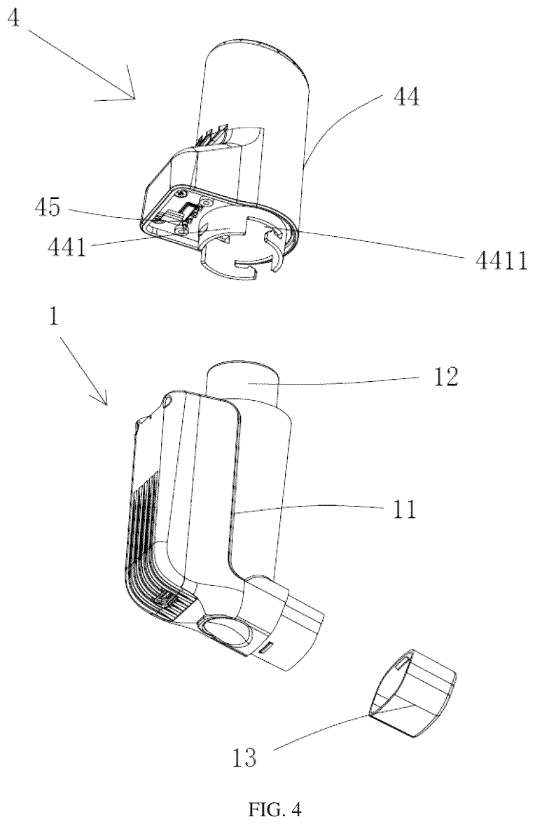

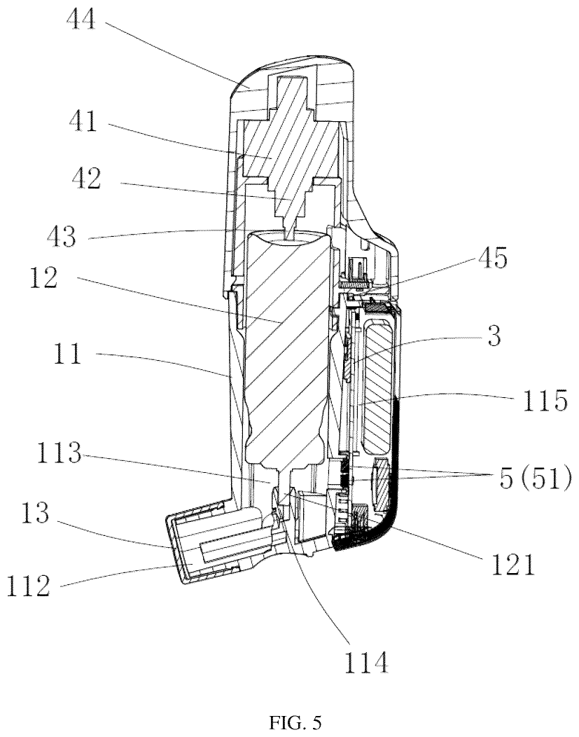

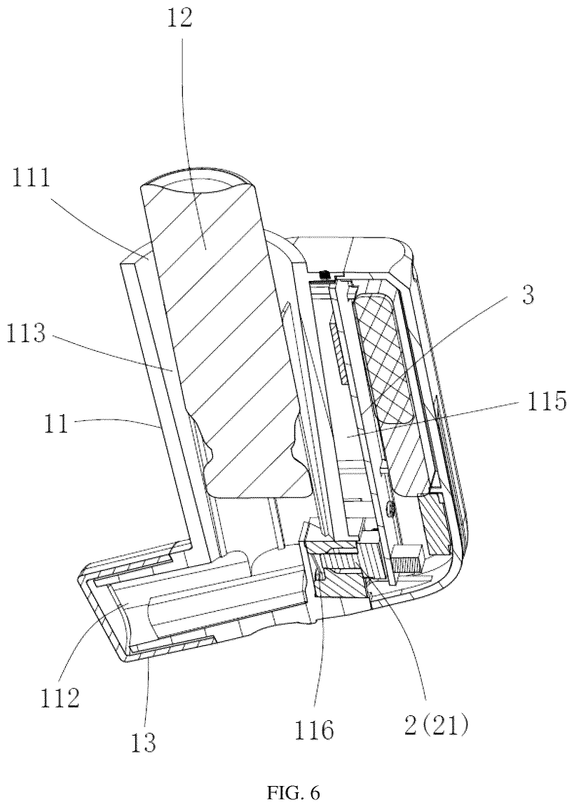

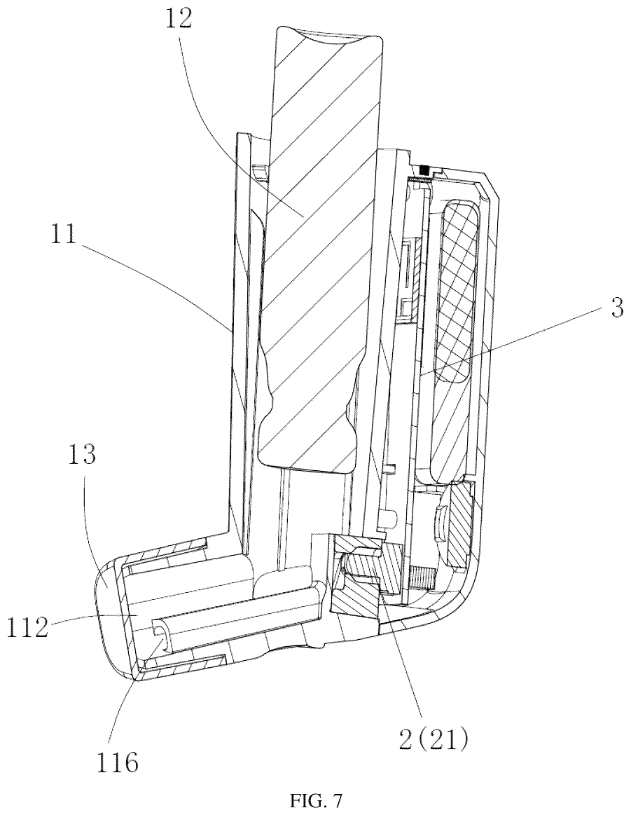

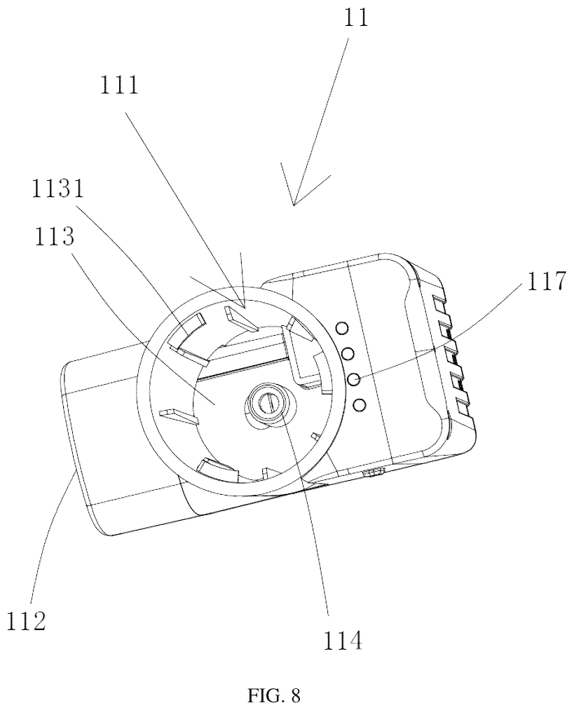

As shown in FIG. 1 to FIG. 8, an automatic drug delivery device for intelligent inhalation medication, including a drug inhaler 1, a drug inhalation action detection unit 2, a main control unit 3 and an automatic pressing unit 4. The drug inhaler 1 is a drug inhaler capable of drug delivery by pressing. More specifically, in this embodiment, the drug inhaler 1 is a pressurized metered-dose inhaler (pMDI), but is not limited to this. The drug inhaler 1 includes an actuator 11 and a canister 12. A front end (lower end in FIG. 5) of the canister 12 has a spray lance 121. The actuator 11 is provided with a chamber 113 having a first end opening 111 and a second end opening 112. The canister 12 is mounted in the chamber 113 from the first end opening 111. A rear end (upper end in FIG. 5) of the canister 12 extends out of the first end opening 111 to facilitate the pressing operation, which is not limited thereto. The second end opening 112 is a drug inhalation opening. During medication, the patient holds the second end opening 112 in the mouth and inhales. A position in the chamber 113 close to the second end opening 112 is provided with a spray guide element 114, and the spray lance 121 of the canister 12 meshes with the spray guide element 114, so that when the canister 12 is pressed from its rear end, the spray lance 121 sprays a dosed amount of drug which flows out through the second end opening 112 and into the patient's mouth. For a more specific structure of the drug inhaler 1, reference may be made to the existing pMDI structure, and details will not be repeated.

In this specific example, the second end opening 112 is provided with an openable cap 13. When the drug inhaler 1 is not in use, the cap 13 covers the second end opening 112 as a protection, which prevents contamination.

The drug inhalation action detection unit 2 is configured to detect whether the drug inhaler 1 is subjected to a drug inhalation action and transmit a drug inhalation action signal to the main control unit 3. The main control unit 3 is configured to correspondingly drive the automatic pressing unit 4 to press the canister 12 of the drug inhaler 1 for drug delivery according to the received drug inhalation action signal.

Specifically, in this embodiment, the drug inhalation action detection unit 2 is preferably realized by a flow rate detection unit 21, which is simple in structure and easy to realize. The flow rate detection unit 21 is configured to detect an inspiratory flow rate of the drug inhaler 1 and transmit an inspiratory flow rate signal to the main control unit 3, and the main control unit 3 is configured to determine whether the drug inhaler 1 is subjected to the drug inhalation action according to the received inspiratory flow rate signal and correspondingly drive the automatic pressing unit 4 to press the canister 12 of the drug inhaler 1 for drug delivery.

In this specific example, the flow rate detection unit 21 is realized by a pressure sensor, which is simple in structure and easy to realize, but is not limited thereto. In some examples, the flow rate detection unit 21 may also be realized by an ultrasonic sensor, a hot-wire flow sensor, a Venturi flow sensor or the like.

Of course, in some examples, the drug inhalation action detection unit 2 may also use another detection unit to detect whether the patient takes a drug inhalation action, for example, an air pressure sensor may be used to detect the change in the air pressure in the chamber 113 so as to determine whether the patient takes the drug inhalation action.

Preferably, in this example, the flow rate detection unit 21 and the main control unit 3 are arranged on the actuator 11, which is more reasonable and compact in structure and easy to realize, but is not limited thereto. In some examples, the flow rate detection unit 21 and the main control unit 3 may also be arranged in other positions, for example, on the automatic pressing unit 4, to form a one-piece structure.

Specifically, in this example, the actuator 11 is provided with a mounting cavity 115. The flow rate detection unit 21 and the main control unit 3 are mounted in the mounting cavity 115. The actuator 11 is provided with a flow rate detection channel 116. The flow rate detection channel 116 communicates with the mounting cavity 115 and the chamber 113 at the first end opening 111. The flow rate detection unit 21 is configured to detect a flow rate of the flow rate detection channel 116 so as to obtain the inspiratory flow rate at the first end opening 111. With this structure, the flow rate detection unit 21 and the main control unit 3 can be easily assembled, and the inspiratory flow rate can be detected accurately without affecting drug inhalation. However, the disclosure is not limited thereto.

The main control unit 3 may be realized by a circuit board having a single-chip microcomputer, which is simple in structure, easy to realize and low in cost. However, the disclosure is not limited thereto.

In this specific example, the automatic pressing unit 4 is realized by a motor 41, which is simple to control, easy to realize and effective in pressing. However, the disclosure is not limited thereto. In some examples, the automatic pressing unit 4 may also be realized by other automatic pressing units such as a cylinder.

More specifically, in this example, the automatic pressing unit 4 includes the motor 41, a reduction box 42 and a screw rod 43. An output shaft of the motor 41 is connected to the screw rod 43 by means of the reduction box 42, and the screw rod 43 is configured to push and press the rear end of the canister 12 of the drug inhaler 1 for drug delivery.

The motor 41 may be realized by a stepping motor, a servo motor or the like.

Preferably, in this specific example, the automatic pressing unit 4 is detachably fixed to the actuator 11 and located outside the first end opening 111 of the actuator 11. In this way, the canister 12 can be replaced easily, and the user may choose whether to mount the automatic pressing unit 4 according to the actual situation. For example, a user with good coordination between the hand and the mouth may choose not to mount the automatic pressing unit 4, but press the canister 12 manually, which is flexible and convenient to use. However, the disclosure is not limited thereto. In some examples, the automatic pressing unit 4 may also be fixedly arranged on the actuator 11 and form a one-piece structure with the actuator 11. For example, the automatic pressing unit 4 may be arranged inside the chamber of the actuator 11, and the main control unit 3 may be arranged inside the automatic pressing unit 4, so that the overall structure is more compact.

The automatic pressing unit 4 further includes a housing 44. The motor 41, the reduction box 42 and the screw rod 43 are fixedly mounted in the housing 44. The housing 44 and the actuator 11 are detachably connected such that the automatic pressing unit 4 is detachably fixed to the actuator 11.

Preferably, the housing 44 and the actuator 11 are detachably connected by means of a rotary snap-fit, which is easy to assemble and disassemble. However, the disclosure is not limited thereto. In some examples, the housing 44 and the actuator 11 may also be connected and fixed by means of other existing detachable structures, such as a threaded connection and a fastening snap-fit.

Specifically, in this example, the housing 44 has a tubular insertion portion 441. The insertion portion 441 is inserted into the chamber 113 at the first end opening 111 of the actuator 11. The insertion portion 441 is provided with an L-shaped groove 4411, and an inner wall of the chamber 113 is provided with a protrusion 1131 matched with the L-shaped groove. The protrusion 1131 and the L-shaped groove 4411 fixedly connect the housing 44 and the actuator 11 by means of a rotary snap-fit. However, the disclosure is not limited thereto. In some examples, the housing 44 may also be detachably connected with the actuator 11 by other rotary snap-fit structures.

Preferably, in this example, the automatic pressing unit 4 is provided with first connecting contacts 45, and the first connecting contacts 45 are electrically connected to the motor 41. The actuator 11 is provided with second connecting contacts 117, and the second connecting contacts 117 are electrically connected to a control output terminal of the main control unit 3. The first connecting contacts 45 and the second connecting contacts 117 contact and are electrically connected with each other, so that the motor 41 is electrically connected to the main control unit 3 by means of the contacting and electrical connection between the first connecting contacts 45 and the second connecting contacts 117. Therefore, when assembling and disassembling the automatic pressing unit 4, there is no need for wiring, which is convenient to operate. However, the disclosure is not limited thereto.

Preferably, the first connecting contacts 45 are realized by spring contact pins, and the second connecting contacts 117 are realized by gold-plated copper posts, which further improves the electrical contact performance, thereby improving the stability and reliability. Of course, in some examples, the first connecting contacts 45 and the second connecting contacts 117 may also be realized by electrical connection structures such as TYPEC male and female connectors and POGO PIN connectors.

Further, in this example, the actuator 11 is further provided with a press-in-place detection unit 5. The press-in-place detection unit 5 is configured to detect whether the canister 12 has been pressed in place, and transmit a detection signal to the main control unit 3. The main control unit 3, after determining that the canister 12 has been pressed in place, controls the automatic pressing unit 4 to stop pressing the canister 12 so as to protect the motor 41 and the canister 12 from damage, thereby further improving the safety and reliability.

In this specific implementation, the press-in-place detection unit 5 is preferably realized by an infrared sensor, which is simple in structure, easy to realize, high in anti-interference ability and low in cost. However, the disclosure is not limited thereto. In some examples, the press-in-place detection unit 5 may also be realized by other existing sensors.

More preferably, the infrared sensor is a reflective infrared sensor 51, which is simpler in structure, easy to realize and low in cost. However, the disclosure is not limited thereto. In some examples, the infrared sensor may also be a through-beam infrared sensor. The reflective infrared sensor 51 is arranged on the actuator 11 and opposite the chamber 113 below the front end of the canister 12. When the canister 12 is pressed down for drug delivery, an outer wall of the front end of the canister 12 moves down to the sensing position of the reflective infrared sensor 51 so as to reflect back infrared rays emitted by the reflective infrared sensor 51, so that the signal received by the reflective infrared sensor 51 is intensified. The main control unit 3 can detect whether the canister 12 has been pressed in place by determining the change of the received signal of the reflective infrared sensor 51.

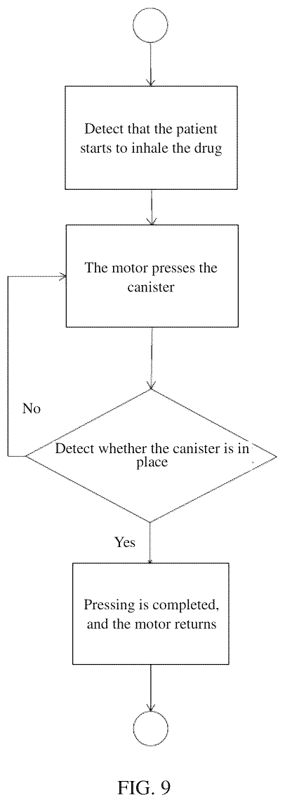

Usage process: When the patient holds the second end opening 112 in the mouth and starts to inhale the drug, the flow rate detection unit 21 detects the inspiratory flow rate and transmits the inspiratory flow rate signal to the main control unit 3, the main control unit 3 outputs a driving signal to drive the motor 41 of the automatic pressing unit 4 to rotate when detecting that the patient starts to inhale the drug according to the received inspiratory flow rate signal, and the motor 41 rotates to drive the reduction box 42 so as to drive the screw rod 43 to push and press down the canister 12, thereby pressing the canister 12. In this way, the disclosure realizes automatic pressing of the canister for drug delivery, and effectively solves the problem that the children and the elderly do not have sufficient pressing force when using a pressurized drug inhaler and the problem that it is likely to press the canister 12 too early or too late during drug inhalation, thereby effectively enhancing the effect of drug therapy and the medication adherence and improving the quality of life. Moreover, the press-in-place detection unit 5 detects whether the canister 12 has been pressed in place in real time. After the canister 12 is pressed in place, the reflective infrared sensor 51 outputs a high level to the main control unit 3, and the main control unit 3 outputs a driving signal such that the motor 41 stops and completes return. The specific process is shown in FIG. 9.

In the design of the driving circuit of the motor 41, another method may be used to protect the pressing process of the automatic pressing unit 4. According to Ohm's law, I=U/R. The main control unit 3 controls a load current ITrip outputted by the driving circuit of the motor 41 to be not greater than ITripMAX, and a magnitude of ITripMAX determines a maximum output torque of the motor 41. An ITripMAX value of the maximum output torque (e.g., 5 kg) may be obtained by means of a laboratory test, and a magnitude of an output voltage of a control lead of the main control unit 3 may be calculated based on this value.

Although the disclosure has been specifically shown and described in connection with the preferred embodiments, it should be understood by those skilled in the art that various changes in form and details can be made without departing from the spirit and scope of the disclosure as defined by the appended claims, and shall all fall within the protection scope of the disclosure.

1. An automatic drug delivery device for intelligent inhalation medication, comprising a drug inhaler, a drug inhalation action detection unit, a main control unit and an automatic pressing unit, wherein the drug inhaler configured to deliver a drug pressing, the drug inhalation action detection unit is configured to detect whether the drug inhaler is subjected to a drug inhalation action and transmit a drug inhalation action signal to the main control unit, and the main control unit is configured to correspondingly drive the automatic pressing unit to press a canister of the drug inhaler for delivery of the drug according to the drug inhalation action signal.

2. The automatic drug delivery device for intelligent inhalation medication according to claim 1, wherein the drug inhalation action detection unit is realized by a flow rate detection unit, the flow rate detection unit being configured to detect an inspiratory flow rate of the drug inhaler and transmit an inspiratory flow rate signal to the main control unit, and the main control unit is configured to correspondingly drive the automatic pressing unit to press the canister of the drug inhaler for delivery of the drug according to the inspiratory flow rate signal.

3. The automatic drug delivery device for intelligent inhalation medication according to claim 2, wherein the drug inhaler comprises an actuator and the canister, the actuator is provided with a chamber having a first end opening and a second end opening, the canister is mounted in the chamber from the first end opening, and the second end opening is a drug inhalation opening.

4. The automatic drug delivery device for intelligent inhalation medication according to claim 3, wherein one of:
the flow rate detection unit comprises a pressure sensor, and the flow rate detection unit and the main control unit are arranged on the actuator; or
the flow rate detection unit, the main control unit and the automatic pressing unit are provided as a one-piece structure.

5. The automatic drug delivery device for intelligent inhalation medication according to claim 4, wherein the automatic pressing unit is detachably fixed to the actuator.

6. The automatic drug delivery device for intelligent inhalation medication according to claim 5, wherein the automatic pressing unit and the actuator are detachably connected by means of a rotary snap-fit, a threaded connection or a fastening snap-fit.

7. The automatic drug delivery device for intelligent inhalation medication according to claim 5, wherein the automatic pressing unit is provided with first connecting contacts, the actuator is provided with second connecting contacts, and the automatic pressing unit is electrically connected to the main control unit by means of contacting and electrical connection between the first connecting contacts and the second connecting contacts.

8. The automatic drug delivery device for intelligent inhalation medication according to claim 4, wherein the automatic pressing unit and the actuator are a one-piece structure.

9. The automatic drug delivery device for intelligent inhalation medication according to claim 3, wherein the automatic pressing unit is detachably fixed to the actuator.

10. The automatic drug delivery device for intelligent inhalation medication according to claim 9, wherein the automatic pressing unit and the actuator are detachably connected by means of a rotary snap-fit, a threaded connection or a fastening snap-fit.

11. The automatic drug delivery device for intelligent inhalation medication according to claim 9, wherein the automatic pressing unit is provided with first connecting contacts, the actuator is provided with second connecting contacts, and the automatic pressing unit is electrically connected to the main control unit by means of contacting and electrical connection between the first connecting contacts and the second connecting contacts.

12. The automatic drug delivery device for intelligent inhalation medication according to claim 3, wherein the automatic pressing unit and the actuator are a one-piece structure.

13. The automatic drug delivery device for intelligent inhalation medication according to claim 4, wherein when the flow rate detection unit and the main control unit are arranged on the actuator, the actuator is provided with a mounting cavity, the flow rate detection unit and the main control unit are mounted in the mounting cavity, the actuator is provided with a flow rate detection channel, the flow rate detection channel communicates with the mounting cavity and the chamber at the first end opening, and the flow rate detection unit is configured to detect a flow rate of the flow rate detection channel.

14. The automatic drug delivery device for intelligent inhalation medication according to claim 1, wherein the automatic pressing unit comprises a motor.

15. The automatic drug delivery device for intelligent inhalation medication according to claim 14, wherein the automatic pressing unit comprises the motor, a reduction box and a screw rod, an output shaft of the motor is connected to the screw rod by means of the reduction box, and the screw rod is configured to push and press the canister of the drug inhaler for delivery of the drug.

16. The automatic drug delivery device for intelligent inhalation medication according to claim 14, wherein the main control unit controls a load current outputted by a driving circuit of the motor to be not greater than a maximum current, a magnitude of the maximum current determines a maximum output torque of the motor, the maximum current of the maximum output torque is obtained by means of a laboratory test, and a magnitude of an output voltage of a control lead of the main control unit is calculated based on the maximum current.

17. The automatic drug delivery device for intelligent inhalation medication according to claim 15, wherein the main control unit controls a load current outputted by a driving circuit of the motor to be not greater than a maximum current, a magnitude of the maximum current determines a maximum output torque of the motor, the maximum current of the maximum output torque is obtained by means of a laboratory test, and a magnitude of an output voltage of a control lead of the main control unit is calculated based on the maximum current.
